(19) United States
(12) Patent Application Publication
(10) Pub. No.: US 2022/0156843 A1
(43) Pub. Date: May 19, 2022

(54) SYSTEMS AND METHODS FOR REAL-TIME ACCESS TO STANDARDIZED MEDICATION INFORMATION

(71) Applicant: KM Initiatives, LLC, McAllen, TX (US)

(72) Inventors: Dimple Patel Desai, McAllen, TX (US); Sagar Arvind Patel, Victoria, TX (US); Darshan Manhar Patel, San Antonio, TX (US); Roshan Arvind Patel, Victoria, TX (US)

(21) Appl. No.: 17/528,527

(22) Filed: Nov. 17, 2021

Related U.S. Application Data
(60) Provisional application No. 63/115,015, filed on Nov. 17, 2020.

(51) Int. Cl. G06Q 40/08; G16H 20/10

(52) U.S. Cl. CPC G06Q 40/08; G16H 20/10

(57) ABSTRACT

The present invention relates to systems and methods for enabling real-time, remote access to up-to-date medication coverage information for medical insurance plans. The inventive concepts disclosed herein enable users to check whether a medication is covered under an insurance plan and identify alternative medications covered by the specified insurance plan. Appropriate dose information for the alternative medications is automatically calculated or determined and provided to users thereby providing users with an opportunity to identify an appropriate medication for a patient in light of the patient's insurance coverage.

### CROSS-REFERENCE TO RELATED APPLICATIONS

This application claims the benefit of U.S. Provisional Application 63/115,015, filed Nov. 17, 2020, titled “SYSTEM AND METHOD FOR VERIFYING MEDICATION COVERAGE AND DETERMINING ALTERNATIVE MEDICATIONS AND DOSAGES COVERED BY INSURANCE PLANS,” which is herein incorporated by reference in its entirety.

### BACKGROUND

### Discussion of the State of the Art

Medical practitioners routinely prescribe medications to patients as part of a treatment plan. However, medical practitioners often do not have insights into whether a prescribed medication is or is not covered by the patient's insurance plan. In instances where the medication is not covered, the cost of the medication to the patient increases significantly, which can lead to lowered compliance/adherence rates and/or significant cost outlay to the patient. In some instances, when a prescribed medication is not covered by the patient's insurance plan, the medical practitioner may be contacted by a pharmacy, an insurance company representative and/or the patient to make alternative prescriptions that may be covered by the patient's insurance plan. This administrative overhead increases the overall costs and inefficiencies associated with treating a patient.

Moreover, currently, there is no system or method by which a medical practitioner can prescribe alternative medication that may or may not be covered by a patient's insurance plan. For example, in any given market, there may be a large number of insurance carriers who may cover different medications under several different policy offerings. Moreover, insurance plans tend to be nuanced and complicated with a variety of different tiers within any single policy offering, with varying levels of coverage requirements, enrollment requirements, etc. Each policy offering (as well as each tier within an offering) may cover some medication but not others depending on the tier or plan that a patient is enrolled in. Moreover, some plans and/or tiers may cover certain medications for certain diagnoses but other plans and/or tiers may cover other medications for the same diagnoses. In addition, insurance plans routinely change the medication that may or may not be covered within various plans or offerings. As such, it is often impossible for any medical practitioner to remain current about medication that may be covered by an insurance plan that a patient may be enrolled in.

In an effort to overcome these challenges, some medical practitioners have resorted to having hospital or clinic staff call various insurance companies to manually or individually identify medication that may be covered by the patient's insurance plan/tier. This enables the medical practitioner and/or the clinic/hospital to improve patient outcomes with respect to medication adherence and reduced costs to the patients. However, this effort is associated with higher administrative costs to the medical clinic/hospital or the medical practitioner. Moreover, this practice provides no guidance in terms of the type and dosage schedule for an alternative medication that should be prescribed for similar efficacy. As such, medical practitioners may incur additional administrative costs in researching the alternatives with similar efficacy.

Indeed, current best practices offer little to no guidance as to which medication should be prescribed to reduce costs to the patients and/or improve compliance/adherence while reducing the administrative costs to the medical providers and the medical system overall.

### SUMMARY

The present invention overcomes these limitations by providing improved systems and methods for prescribing medication that is covered by a patient's insurance plan. More specifically, the systems and methods of the present inventions enable a medical practitioner to automatically determine whether a medication is covered by the patient's insurance plan. If a medication is not covered, the systems and methods of the present inventions identify alternative medications and the dosage regimen associated with the alternative medication that have similar efficacy as the original medicine.

### BRIEF DESCRIPTION OF THE DRAWING FIGURES

The accompanying drawings illustrate several embodiments and, together with the description, serve to explain the principles of the invention according to the embodiments. It will be appreciated by one skilled in the art that the particular arrangements illustrated in the drawings are merely exemplary and are not to be considered as limiting of the scope of the invention or the claims herein in any way.

FIG. 1 illustrates an exemplary system for determining alternative medications and dosages in accordance with an exemplary embodiment of the invention

FIG. 2 illustrates one embodiment of the components of a system for determining alternative medications and dosages in accordance with an exemplary embodiment of the invention.

FIG. 3A illustrates one embodiment of a process for determining medication coverage and identifying alternative medications and dosages in accordance with an exemplary embodiment of the invention.

FIG. 3B illustrates an exemplary user interface for use in determining medication coverage and identifying alternative medications and dosages in accordance with an exemplary embodiment of the invention.

FIG. 4 illustrates one embodiment of the computing architecture that supports an embodiment of the inventive disclosure.

FIG. 5 illustrates components of a system architecture that supports an embodiment of the inventive disclosure.

FIG. 6 illustrates components of a computing device that supports an embodiment of the inventive disclosure.

FIG. 7 illustrates components of a computing device that supports an embodiment of the inventive disclosure.

### DETAILED DESCRIPTION

The inventive system and method (hereinafter sometimes referred to more simply as “system” or “method”) described herein significantly reduces the time and effort required by a practitioner to find alternative medications when a prescribed medication is not covered by a patient's insurance plan. In addition, the disclosed system and method lead to a more efficient prescription filling process by reducing the need for pharmacies and/or patients to contact a practitioner about an alternative medication allowing the patient to more readily receive the medication they need.

One or more different embodiments may be described in the present application. Further, for one or more of the embodiments described herein, numerous alternative arrangements may be described; it should be appreciated that these are presented for illustrative purposes only and are not limiting of the embodiments contained herein or the claims presented herein in any way. One or more of the arrangements may be widely applicable to numerous embodiments, as may be readily apparent from the disclosure. In general, arrangements are described in sufficient detail to enable those skilled in the art to practice one or more of the embodiments, and it should be appreciated that other arrangements may be utilized and that structural, logical, software, electrical and other changes may be made without departing from the scope of the embodiments. Particular features of one or more of the embodiments described herein may be described with reference to one or more particular embodiments or figures that form a part of the present disclosure, and in which are shown, by way of illustration, specific arrangements of one or more of the aspects. It should be appreciated, however, that such features are not limited to usage in the one or more particular embodiments or figures with reference to which they are described. The present disclosure is neither a literal description of all arrangements of one or more of the embodiments nor a listing of features of one or more of the embodiments that must be present in all arrangements.

Headings of sections provided in this patent application and the title of this patent application are for convenience only and are not to be taken as limiting the disclosure in any way.

Devices that are in communication with each other need not be in continuous communication with each other, unless expressly specified otherwise. In addition, devices that are in communication with each other may communicate directly or indirectly through one or more communication means or intermediaries, logical or physical.

A description of an aspect with several components in communication with each other does not imply that all such components are required. To the contrary, a variety of optional components may be described to illustrate a wide variety of possible embodiments and in order to more fully illustrate one or more embodiments. Similarly, although process steps, method steps, algorithms or the like may be described in a sequential order, such processes, methods and algorithms may generally be configured to work in alternate orders, unless specifically stated to the contrary. In other words, any sequence or order of steps that may be described in this patent application does not, in and of itself, indicate a requirement that the steps be performed in that order. The steps of described processes may be performed in any order practical. Further, some steps may be performed simultaneously despite being described or implied as occurring non-simultaneously (e.g., because one step is described after the other step). Moreover, the illustration of a process by its depiction in a drawing does not imply that the illustrated process is exclusive of other variations and modifications thereto, does not imply that the illustrated process or any of its steps are necessary to one or more of the embodiments, and does not imply that the illustrated process is preferred. Also, steps are generally described once per aspect, but this does not mean they must occur once, or that they may only occur once each time a process, method, or algorithm is carried out or executed. Some steps may be omitted in some embodiments or some occurrences, or some steps may be executed more than once in a given aspect or occurrence.

When a single device or article is described herein, it will be readily apparent that more than one device or article may be used in place of a single device or article. Similarly, where more than one device or article is described herein, it will be readily apparent that a single device or article may be used in place of the more than one device or article.

The functionality or the features of a device may be alternatively embodied by one or more other devices that are not explicitly described as having such functionality or features. Thus, other embodiments need not include the device itself.

Techniques and mechanisms described or referenced herein will sometimes be described in singular form for clarity. However, it should be appreciated that particular embodiments may include multiple iterations of a technique or multiple instantiations of a mechanism unless noted otherwise. Process descriptions or blocks in figures should be understood as representing modules, segments, or portions of code which include one or more executable instructions for implementing specific logical functions or steps in the process. Alternate implementations are included within the scope of various embodiments in which, for example, functions may be executed out of order from that shown or discussed, including substantially concurrently or in reverse order, depending on the functionality involved, as would be understood by those having ordinary skill in the art.

### Conceptual Architecture

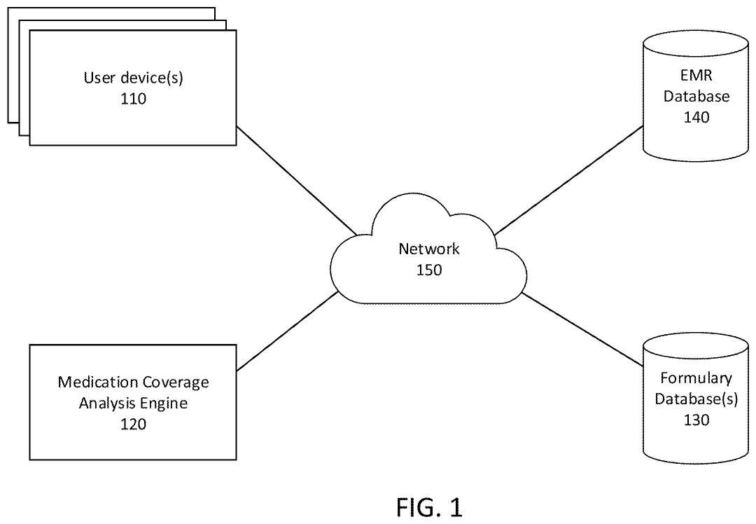

FIG. 1 illustrates an exemplary embodiment of a system for enabling real-time access to standardized medication information according to one embodiment. The system includes a user device(s) 110 which includes a medication coverage analysis engine 120, formulary database(s) 130, an electronic medical records (EMR) database(s) 140 (also sometimes referred to as an electronic health record (EHR)), and a network 150 over which the various systems communicate and interact. The system is configured to provide a plurality of users with remote access over a network so that any of the users can provide medication and insurance information through user device(s) 110 (e.g. via a graphical user interface of the user device(s) 110) and receive up to date medication coverage and alternative medication information in real-time from at least one of the medication coverage analysis engine 120, formulary database(s) 130, and electronic medical records (EMR) database(s) 140. The various computing devices described herein are exemplary and for illustration purposes only. The system may be reorganized or consolidated, as understood by a person of ordinary skill in the art, to perform the same tasks on one or more other servers or computing devices without departing from the scope of the invention.

The user device(s) 110 generally comprises a computer or computing device as discussed in more detail below. The user device(s) 110 may be used to provide information related to medication(s) and insurance information and receive information associated with coverage of the medication(s) under insurance plans as well as alternative medications. For example, a prescription for a given medication and information associated with a patient's insurance plan may be entered into the user device via a user interface and relayed to a medication coverage analysis engine 120 via network 150 for a determination if the prescribed medication will be covered. In one aspect, user device(s) 110 may be used by a licensed clinician with the authority to prescribe medications such as physicians, physician's assistants, nurse practitioners, and the like in order to check coverage of a medication and/or prescribe a desired or alternative medication. In one aspect, user device(s) 110 may be used be a patient to submit medication information in order to determine if a medication is covered by their insurance plan. In one aspect, user device(s) 110 may be used by personnel associated with a prescribing clinician in order to check coverage of a medication under a particular insurance plan. In one aspect user device(s) 110 may be used by medication dispensing and fulfillment entities such as pharmacies in order to check coverage of a medication under a particular insurance plan.

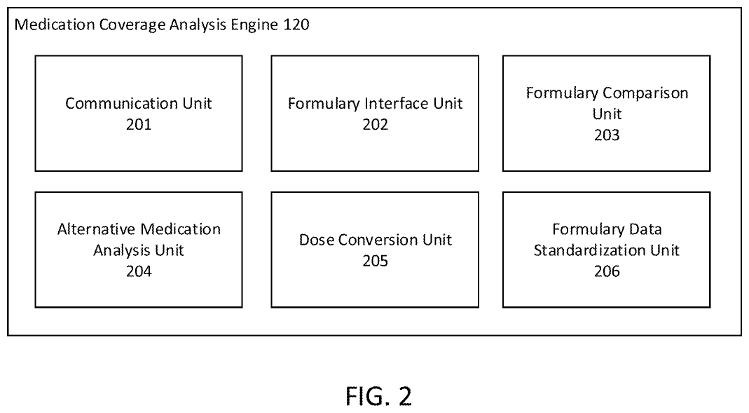

The medication coverage analysis engine 120 receives medication information and medical insurance information, processes this information, and outputs a result associated with insurance plan coverage for the medication. Generally, the medication coverage analysis engine obtains medication information and medical insurance information from a user device 110 via network 150 and processes this information to determine if the medication is covered in association with the medical insurance plan. If the medication is covered, the user is provided with an indication of coverage and in some applications, a prescription for the medication is generated and submitted to an appropriate medication dispenser or medication fulfillment entity. If the medication is not covered, the user is provided with an indication of non-coverage and provided with alternative medications and converted dosages which are covered in association with the medical insurance information. This medication coverage analysis may involve obtaining formulary data such as from formulary database(s) 130 via network 150. The medication coverage analysis engine 120 is discussed in more detail below under the discussion of FIG. 2.

The medication coverage analysis engine 120 may take the form of a cloud-based, web-based, remote server-based or other network-based engine accessed remotely via network 150 according to FIG. 1, or, although not depicted, an app-based configuration incorporated into user device(s) 110. In one aspect, medication coverage analysis engine 120 receives at least one of medication and patient insurance information via user device(s) 110. In one aspect, medication coverage analysis engine 120 receives at least one of medication and patient insurance information via an EMR database. The EMR database may also periodically pull appropriate formulary information related to a patient's insurance plan coverage from the formulary databases so this information is readily available and updated within the EMR database. Alternatively, while not depicted, the medication coverage analysis engine 120 may be incorporated into EMRs. For example, this may be integrated into the EMR via necessary programming code for the EMR, in the form of a plug-in or add-on application, a hyperlink to the medication coverage analysis engine 120, or other suitable form as would be recognized by one of ordinary skill in the art. Any suitable form of integration, incorporation, and interfacing with any EMR may be used in any embodiment of the inventive concepts disclosed herein.

The formulary database(s) 130 store information about medication coverages. This may include a list of medications and dosages and their associated coverage under a plurality of different insurance plans. Alternate formats of this database are possible such as a list of different insurance plans and the medications covered under each of these insurance plans. Although not depicted in the figures, the formulary databases 130 may be incorporated into other components of the system. For example, the formulary databases 130 may be incorporated into the medication analysis engine 120 (in any of a network-based, app-based, EMR or EHR integrated configuration), or as part of an EMR or EHR database such that they are readily accessible as needed. The formulary database(s) 130 may be created by obtaining formulary information for a plurality of medical insurance plans, such as by scanning published formulary information, downloading formulary information from websites such as insurance company websites, through API calls to obtain formulary information from a variety of external sources, via manual data entry, and via an insurance company pushing updated formulary information to the formulary database(s) 130 etc.

The obtained formulary information may be converted into a standardized format, such as by an associated processor which may be part of or distinct from the medication coverage analysis engine 120. Converting the formulary information into a standardized format may comprise the use of a standardization filter in order to extract the relevant information, such as covered or non-covered medication names, classes, dose information, diagnosis information, benefit levels, etc., from different formulary sources. The standardized format of the obtained formulary information may then be stored in the formulary database(s) 130 so that evaluation of formulary information can be performed in a systematic manner in order to determine medication coverages. The formulary database(s) 130 are updated at periodic intervals such as regularly occurring time frames (e.g. annually, bi-annually, monthly, etc.) or whenever new formulary information is released or published by a medical insurance company.

User device(s) 110 include, generally, a computer or computing device including functionality for communicating (e.g., remotely) over a network 150. Data may be collected from user devices 110, and data requests may be initiated from each user device 110. User device(s) 110 may be a server, a desktop computer, a laptop computer, personal digital assistant (PDA), an in- or out-of-car navigation system, a smart phone or other cellular or mobile phone, or mobile gaming device, among other suitable computing devices. User devices 110 may execute one or more client applications, such as a web browser (e.g., Microsoft Windows Internet Explorer, Mozilla Firefox, Apple Safari, Google Chrome, and Opera, etc.), or a dedicated application to submit user data, or to make prediction queries over a network 150.

In particular embodiments, each user device 110 may be an electronic device including hardware, software, or embedded logic components or a combination of two or more such components and capable of carrying out the appropriate functions implemented or supported by the user device 110. For example and without limitation, a user device 110 may be a desktop computer system, a notebook computer system, a netbook computer system, a handheld electronic device, or a mobile telephone. The present disclosure contemplates any user device 110. A user device 110 may enable a network user at the user device 110 to access network 110. A user device 110 may enable its user to communicate with other users at other user devices 110.

A user device 110 may have a web browser, such as MICROSOFT INTERNET EXPLORER, GOOGLE CHROME or MOZILLA FIREFOX, and may have one or more add-ons, plug-ins, or other extensions, such as TOOLBAR or YAHOO TOOLBAR. A user device 110 may enable a user to enter a Uniform Resource Locator (URL) or other address directing the web browser to a server, and the web browser may generate a Hyper Text Transfer Protocol (HTTP) request and communicate the HTTP request to server. The server may accept the HTTP request and communicate to the user device 110 one or more Hyper Text Markup Language (HTML) files responsive to the HTTP request. The user device 110 may render a web page based on the HTML files from server for presentation to the user. The present disclosure contemplates any suitable web page files. As an example and not by way of limitation, web pages may render from HTML files, Extensible Hyper Text Markup Language (XHTML) files, or Extensible Markup Language (XML) files, according to particular needs. Such pages may also execute scripts such as, for example and without limitation, those written in JAVASCRIPT, JAVA, MICROSOFT SILVERLIGHT, combinations of markup language and scripts such as AJAX (Asynchronous JAVASCRIPT and XML), and the like. Herein, reference to a web page encompasses one or more corresponding web page files (which a browser may use to render the web page) and vice versa, where appropriate.

The user device 110 may also include an application that is loaded onto the user device 110. The application 110 obtains data from the network 110 and displays it to the user within the application interface.

Exemplary user devices are illustrated in some of the subsequent figures provided herein. This disclosure contemplates any suitable number of user devices, including computing systems taking any suitable physical form. As example and not by way of limitation, computing systems may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, or a combination of two or more of these. Where appropriate, the computing system may include one or more computer systems; be unitary or distributed; span multiple locations; span multiple machines; or reside in a cloud, which may include one or more cloud components in one or more networks. Where appropriate, one or more computing systems may perform without substantial spatial or temporal limitation one or more steps of one or more methods described or illustrated herein. As an example, and not by way of limitation, one or more computing systems may perform in real time or in batch mode one or more steps of one or more methods described or illustrated herein. One or more computing system may perform at different times or at different locations one or more steps of one or more methods described or illustrated herein, where appropriate.

Network cloud 150 generally represents a network or collection of networks (such as the Internet or a corporate intranet, or a combination of both) over which the various components illustrated in FIG. 1 (including other components that may be necessary to execute the system described herein, as would be readily understood to a person of ordinary skill in the art). In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network 150 or a combination of two or more such networks 150. One or more links connect the systems and databases described herein to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable network 150, and any suitable link for connecting the various systems and databases described herein.

The network 150 connects the various systems and computing devices described or referenced herein. In particular embodiments, network 150 is an intranet, an extranet, a virtual private network (VPN), a local area network (LAN), a wireless LAN (WLAN), a wide area network (WAN), a metropolitan area network (MAN), a portion of the Internet, or another network or a combination of two or more such networks 150. The present disclosure contemplates any suitable network 150.

One or more links couple one or more systems, engines or devices to the network 150. In particular embodiments, one or more links each includes one or more wired, wireless, or optical links. In particular embodiments, one or more links each includes an intranet, an extranet, a VPN, a LAN, a WLAN, a WAN, a MAN, a portion of the Internet, or another link or a combination of two or more such links. The present disclosure contemplates any suitable links coupling one or more systems, engines or devices to the network 150.

In particular embodiments, each system or engine may be a unitary server or may be a distributed server spanning multiple computers or multiple datacenters. Systems, engines, or modules may be of various types, such as, for example and without limitation, web server, news server, mail server, message server, advertising server, file server, application server, exchange server, database server, or proxy server. In particular embodiments, each system, engine or module may include hardware, software, or embedded logic components or a combination of two or more such components for carrying out the appropriate functionalities implemented or supported by their respective servers. For example, a web server is generally capable of hosting websites containing web pages or particular elements of web pages. More specifically, a web server may host HTML files or other file types, or may dynamically create or constitute files upon a request, and communicate them to clients devices or other devices in response to HTTP or other requests from clients devices or other devices. A mail server is generally capable of providing electronic mail services to various client devices or other devices. A database server is generally capable of providing an interface for managing data stored in one or more data stores.

In particular embodiments, one or more data storages may be communicatively linked to one or more servers via one or more links. In particular embodiments, data storages may be used to store various types of information. In particular embodiments, the information stored in data storages may be organized according to specific data structures. In particular embodiments, each data storage may be a relational database. Particular embodiments may provide interfaces that enable servers or clients to manage, e.g., retrieve, modify, add, or delete, the information stored in data storage.

The system may also contain other subsystems and databases, which are not illustrated in FIG. 1, but would be readily apparent to a person of ordinary skill in the art. For example, the system may include databases for storing data, storing features, storing outcomes (training sets), and storing models. Other databases and systems may be added or subtracted, as would be readily understood by a person of ordinary skill in the art, without departing from the scope of the invention.

### Medication Coverage Analysis Engine

FIG. 2 illustrates an exemplary embodiment of the medication coverage analysis engine 120. The engine comprises a communication unit 201, formulary interface unit 202, formulary comparison unit 203, alternative medication analysis unit 204, dose conversion unit 205, formulary data standardization unit 206, and may optionally include formulary databases 130 as discussed above with respect to FIG. 1.

The communication unit 201 obtains medication information and medical insurance information, relays this information to other units of the medication coverage analysis engine 120 for processing, communicates results of the processing to the network 150 and ultimately to a user device 110, and may communicate with a medication dispenser or medication fulfillment entity, such as a pharmacy.

The formulary interface unit 202 obtains information from formulary data sources such as formulary database(s) 130 associated with medication coverage information or via other formulary data acquisition approaches as discussed above with respect to FIG. 1 and how the formulary information is obtained in association with formulary database(s) 130. For example, when medication information is received along with medical insurance information, the formulary interface unit 202 obtains formulary information specific to the medical insurance information. In one aspect, the formulary interface unit 202 retrieves formulary information from formulary data sources such as formulary databases and/or published formulary information. In one aspect, the formulary interface unit 202 obtains formulary information via communication with insurance companies such as via an insurance company pushing updated formulary information to the medication coverage analysis engine 120 or via an API call from the medication coverage analysis engine 120 requesting up to date formulary information from an insurance provider. This data may be used by the formulary comparison unit 203 in order to determine coverage as described below.

The formulary comparison unit 203 obtains medication information and formulary information specific to the medical insurance information. The formulary information specific to the medical insurance information may be obtained in a standardized format. The formulary comparison unit 203 compares the medication information with the information received from the formulary databases 130 to determine if the medication is covered by the insurance plan or if an alternative medication is required. If the medication is covered by the patient's insurance plan, the formulary comparison unit 203 determines such and communicates such to a user device and a medication filling entity via the communication unit 201. If the medication is not covered by the patient's insurance plan, the formulary comparison unit communicates with the alternative medication analysis unit 204 and dose conversion unit 205 to determine a suitable alternative medication covered by the patient's insurance plan.

The alternative medication analysis unit 204 receives the obtained medication information and formulary information associated with the medical insurance information and determines at least one alternative medication. For example, when the formulary comparison unit 203 determines that obtained medication information is not covered under a given insurance plan, the alternative medication analysis unit 204 may obtain the obtained medication information and formulary coverage information associated with the medical insurance information. The alternative medication analysis unit 204 may use this information to determine at least one suitable alternative medication to the obtained medication which is covered in association with the obtained medical insurance information. This may include identifying at least one alternative medication of the same class as the obtained medication and using the dose conversion unit 205 to determine the appropriate equivalent dose information. For example, in one embodiment, the alternative medication analysis unit 204 receives a medication that a medical practitioner wants to prescribe, and identifies the class that the medication is in (a variety of different databases may be used and/or a lookup function may be employed to identify the class of a medication). Once the class is identified, the alternative medication analysis unit 204 identifies other medications within the same class. The alternative medication analysis unit 204 may interface with the formulary comparison unit 203 to identify other medications in the same class as the medication that the practitioner wants to prescribe that are covered by the patient's insurance plan/tier. Once other medication in the same class is identified as being covered by the patient's insurance plan/tier, the medication analysis unit 204 interfaces with the dose conversion unit 205 to identify an appropriate equivalent dosage for the alternative medicine that should be prescribed.

The dose conversion unit 205 obtains the medication information and alternative medication information and determines equivalent dose information for each of the at least one alternative medications. Dose conversion may comprise use of lookup or reference tables to find the equivalent dose, may be found in the formulary information, or may be calculated based on information associated with each medication and known conversion calculations. In one aspect, the dose conversion is performed automatically, by a processor, in response to receiving or otherwise obtaining medication information and medical insurance information. Once alternative medications and dose information have been determined, this information may be provided to user device(s) 110 in real-time via communication unit 201 and network 150.

In one aspect, the medication coverage engine 120 comprises a formulary data standardization unit 206 which obtains formulary information for a plurality of medical insurance plans, such from scanning published formulary information, downloading formulary information from websites such as insurance company websites, through API calls to obtain formulary information from a variety of external sources, via manual data entry, etc. and then converts the obtained formulary information may be converted into a standardized format. Converting to a standardized format may comprise the use of a standardization filter in order to extract the relevant information, such as covered or non-covered medication names, classes, dose information, diagnosis information, benefit levels, etc. Converting to a standardized format may comprise making relevant data conform to a similar format, such as categorizing or labelling the data, applying a particular structure to the data, and ordering the data in a particular way. Once converted, the standardized format of the data may be stored in a formulary database(s) which may either be an external component such as depicted in FIG. 1 or incorporated into FIG. 2 (not shown). The formulary data standardization unit 206 may periodically obtain and standardize new formulary information to be stored in formulary database(s) such as at set time intervals (e.g. annually, bi-annually, monthly, etc.) or whenever a medical insurance company publishes or otherwise releases updated formulary information.

The medication coverage analysis engine 120 discussed above may be integrated into or otherwise communicate with different EMR or EHR systems. In one aspect, the medication coverage analysis engine may be deployed to existing EMR or EHR systems via deployable universal code provided to EMRs or via EMR specific code. In one aspect, an EMR or EHR system may be used as an interface to communicate medication information and medical insurance information, such as information associated with a particular patient to the medication coverage analysis engine 120. In these scenarios, the medication coverage analysis engine 120 is configured to translate or convert the incoming information from different EMR or EHR systems into a universal standard for further processing by the medication coverage analysis engine 120.

### Process for Determining Medication Coverage and Identifying Alternative Medications

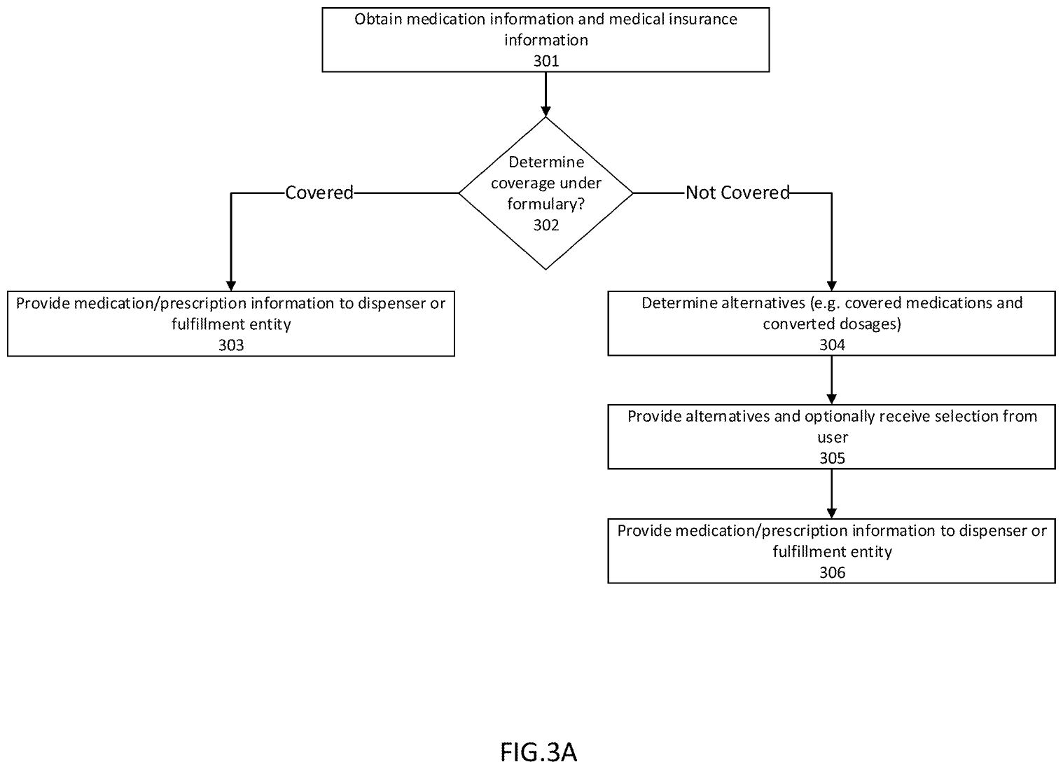

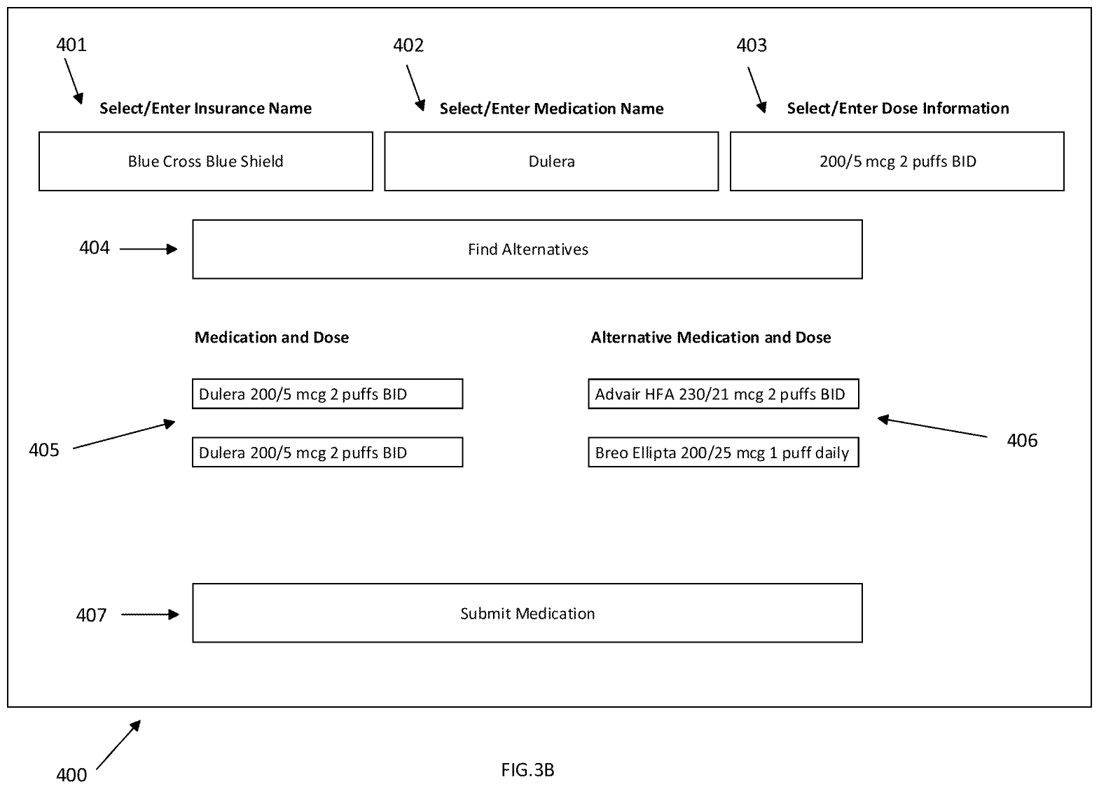

FIG. 3A illustrates an exemplary process for determining medication coverage and identifying alternative medications and dosages according to an embodiment of the invention. The process comprises obtaining medication information and medical insurance information 301, determining coverage under formulary information associated with the medical insurance information 302, providing covered medication or prescription information

At step 301, medication information and medical insurance information are obtained. This information may be obtained in a variety of ways including, but not limited to, from user devices associated with a user such as from a patient, a prescribing practitioner, healthcare staff/personnel, pharmacists, and pharmacy staff/personnel, or from electronic medical records (EMRs) or electronic health records (EHRs). In one aspect, the medication information may include at least one of a medication name, medication class, corresponding dose information, and diagnosis information the medication is intended to treat. In one aspect, it may be sufficient to obtain only one of these pieces of information. In one aspect, depending on the circumstances, it may be necessary to obtain a plurality of these listed items or even additional information. For example, for a given insurance plan, the plan may not cover certain medications, classes of medication, certain dose characteristics, or a given medication for a certain diagnosis. Dose information includes, but is not limited to dose, dosage, dosage form, and dosage regimen. Diagnosis information comprises at least one of a diagnosis name and a diagnosis code associated with the need for the medication. Medical insurance information comprises at least one of the name of an insurance plan, such as a plan associated with a particular patient, insurance plan type (e.g. PPO, HMO, etc.), if applicable, a benefit or coverage level (e.g. basic, standard, etc.), if applicable, and a medical insurance identifier (e.g. identification number) associated with a patient. In one aspect, the obtaining of medication information and medical insurance information may be done as part of investigating medication coverage such as to plan a potential treatment regimen that will be covered by a given patient's medical insurance in order to increase likelihood of patient compliance with obtaining the desired medication as opposed to scenarios where a patient foregoes obtaining a medication because their insurance does not cover it. In one aspect, the obtaining of medication information and medical insurance information may be done as part of a medication prescribing process such that, via the subsequent steps, a check can be performed to determine if the medication being prescribed will be covered by the patient's insurance, and when it is not, the prescriber is presented with alternative medications and afforded the opportunity to modify the prescription as deemed appropriate by the prescriber.

At step 302, a determination is made as to whether the obtained medication information is covered in association with the obtained medical insurance information. The determination of coverage at step 302 may be accomplished by a medication coverage analysis engine 120 such as the one described above. For example, the process may comprise determining whether the obtained medication information is covered by the obtained medical insurance information by performing a lookup operation in a formulary database and/or retrieved or otherwise obtained formulary information. The obtained or retrieved formulary information may be obtained or retrieved as discussed above with respect to FIG. 1 and FIG. 2 and may comprise formulary information converted to a standardized format and stored in the standardized format.

In one aspect, to determine coverage, the process may comprise identifying insurance specific formulary information which is formulary information associated with the obtained medical insurance information. In one aspect, identifying insurance specific formulary information comprises identifying formulary information associated with the obtained medical insurance information from stored formulary information associated with a plurality of medical insurance plans which has been stored in a standardized format. This stored formulary information may be obtained and stored as discussed above with respect to FIG. 1. Next, the medication information and insurance specific formulary information are evaluated, by a processor, to determine the presence or absence of the obtained medication information. For example, the obtained medication information is compared with insurance specific formulary information to determine the presence or absence of the medication information in the insurance specific formulary information. If the medication information is present or listed in the formulary information or formulary database, then the determination is made that the medication information is covered. If the medication information is absent or not listed in the formulary information or formulary database, then the determination is made that the medication information is not covered. The next steps in the process may be dependent on the result of this coverage determination.

If, at step 302, a determination is made that the obtained medication information is not covered in association with the obtained insurance information, the process proceeds to step 304 to determine alternative medications and dosages that are covered by the obtained insurance information by identifying alternative medication(s) to the obtained medication information that is/are present in the insurance specific formulary information. This may be accomplished in the same or a similar fashion as in the medication coverage analysis engine 120 described above. In one aspect, the process comprises identifying medications of the same class as the obtained medication information which are covered by the patient's insurance plan. For each identified equivalent medication in the same class, equivalent dose information may be determined by referencing conversion tables in order to determine an appropriate dosage and dosing timing (i.e. a dosing schedule) for each equivalent medication. In one aspect, the formulary information may directly indicate a substitute or alternative medication and corresponding dose information without the need for class identification and dose conversion.

Once alternative medications and equivalent dose information have been determined, a user is provided with the alternative medication and dose information in real-time or near real-time, at step 305. In one aspect, this comprises providing the alternative medication and dose information such that it is accessible by one or more user devices thereby providing at least one user with real-time remote access to standardized medication and insurance coverage information. For example, this may comprise providing the information such that at least one of a patient, prescriber and pharmacist are all able to access the information from their respective user device(s). As another example, in a scenario where the user is a prescribing practitioner, such as a physician, the practitioner may receive a notification on at least one user device (e.g. a smartphone, a computer, a tablet, a computing device associated with an EMR, etc.) indicating that a prescribed medication for a given patient is not covered by the patient's insurance. The user device associated with the practitioner may display the obtained medication information along with alternative medications and dose information for each covered alternative as identified in step 304 above.

Upon receiving and viewing the alternative medication and dose information, a user may be presented with an interface to allow the practitioner to select either the original medication information (i.e. the obtained or prescribed medication information) or an alternative medication and dose information from the information generated in step 304. Upon selection of the desired medication using a user device, the user may then elect to submit said selection for fulfillment of the corresponding prescription, such as submitting the prescription information to an indicated medication dispenser or fulfillment entity such as a pharmacy. One example of a user interface for receiving and selecting an alternative medication and dose information is presented in FIG. 3B as discussed in more detail below.

This is one example wherein the process may be used in the prescription process to make prescription decisions and changes in real time based on coverage information being made readily available to the prescribing practitioner. Other applications of the process include investigative or planning approaches wherein the alternative medication information may be used to identify covered and non-covered medication options for a given patient or insurance plan and the process does not necessarily require selection of a medication and submission of a prescription as discussed above. Furthermore, regardless of whether a medication is determined as covered or not covered, alternative medications may still be determined and provided to users such that users are enabled to access up to date alternative medication coverage and dose information even when a preferred or identified medication is covered.

At step 306, if the user elects to submit the selected medication information, a corresponding prescription is generated and transmitted or otherwise communicated to a medication dispenser or medication fulfillment entity (e.g. a pharmacy). In one aspect, the user (e.g. prescribing practitioner) may be notified of successful transmission to the indicated medication dispenser of medication fulfillment entity.

Returning to step 302, if the determination indicates that the obtained medication information is covered in association with the obtained medical insurance information, the process proceeds to step 303 wherein the obtained medication information is transmitted or otherwise communicated to a designated medication dispenser or fulfillment entity (e.g. a pharmacy) for processing. In one aspect, the user (e.g. the prescribing practitioner) may be notified of confirmed coverage and successful transmission to the medication dispenser or medication fulfillment entity. In this particular example, the process is primarily directed at aiding a prescribing practitioner during the medication prescribing process, however the process includes other alternatives which do not require providing medication information to a dispenser or fulfillment entity. For example, the process may comprise simply providing an indication to a user that the obtained medication information is covered in association with the obtained medical insurance information. Such scenarios may be beneficial when using the inventive concepts disclosed herein for planning purposes or investigation of insurance plans and need not necessarily be implemented in a fashion that requires prescribing medications and providing medication information to fulfillment entities.

While the above process is focused on whether a medication is covered or not covered, a similar process may be implemented in order to evaluate cost effectiveness of different medications for an individual based on their particular insurance. Identifying a more cost friendly alternative medication, may increase patient compliance since a patient may more easily obtain the medication due to better affordability. For example, even when step 302 determines a medication is covered, although not depicted, a check for alternative medications as in step 304 and presentation of alternatives to a user (e.g. prescribing practitioner) as in step 305 may also be performed under the path labeled “Covered” of FIG. 3A prior to step 303. Such approaches may be applicable in situations where medical insurance plans have different tiers of medication coverage wherein some medications are covered at different amounts and patient out of pocket costs are dependent on which tier the prescribed medication falls within.

FIG. 3B illustrates an exemplary user interface 400 for use in association with the above process and a medication coverage analysis engine. The user interface 400 comprises data entry elements 401, 402, 403, element 404 to initiate identification of alternative medications, elements 405 and 406 to display and receive selection of provided and alternative medications and corresponding dosages, and element 407 to submit a selected medication for further processing.

At data entry elements 401, 402, and 403, a user enters, or selects from a list, insurance information at element 401, medication name at 402, and dose information at 403. Once information has been entered in elements 401, 402 and 403, a user selects (e.g. clicks, taps, etc.) element 404 (e.g. a button) which initiates a process to identify medication coverage and determine alternative medications and corresponding dosages. This identification and determination of alternative medications may be done as described above with respect to FIG. 2 and FIG. 3A. Element 405 displays the provided medication and dose information alongside each identified alternative medication and corresponding dosage which are displayed at element 406. In one aspect, the medications and dosages displayed at 405 and 406 are interactive elements such as buttons that can be clicked, tapped, or otherwise selected such that a user may choose any of the displayed medication options. Upon selection of an option from 405 or 406, a user may then select (e.g. click, tap, etc.) the submit medication element 407 (e.g. button) which initiates the generation and transmission of a corresponding prescription for the selected medication to an indicated medication dispenser or fulfillment entity.

### Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card. For example, the above described medication coverage analysis engine 120 and its sub-units may be implemented as hardware or a combination of hardware and software as described herein.

Software/hardware hybrid implementations of at least some of the embodiments disclosed herein may be implemented on a programmable network-resident machine (which should be understood to include intermittently connected network-aware machines) selectively activated or reconfigured by a computer program stored in memory. Such network devices may have multiple network interfaces that may be configured or designed to utilize different types of network communication protocols. A general architecture for some of these machines may be described herein in order to illustrate one or more exemplary means by which a given unit of functionality may be implemented. According to specific embodiments, at least some of the features or functionalities of the various embodiments disclosed herein may be implemented on one or more general-purpose computers associated with one or more networks, such as for example an end-user computer system, a client computer, a network server or other server system, a mobile computing device (e.g., tablet computing device, mobile phone, smartphone, laptop, or other appropriate computing device), a consumer electronic device, a music player, or any other suitable electronic device, router, switch, or other suitable device, or any combination thereof. In at least some embodiments, at least some of the features or functionalities of the various embodiments disclosed herein may be implemented in one or more virtualized computing environments (e.g., network computing clouds, virtual machines hosted on one or more physical computing machines, or other appropriate virtual environments). Any of the above mentioned systems, units, modules, engines, components or the like may be and/or comprise hardware and/or software as described herein.

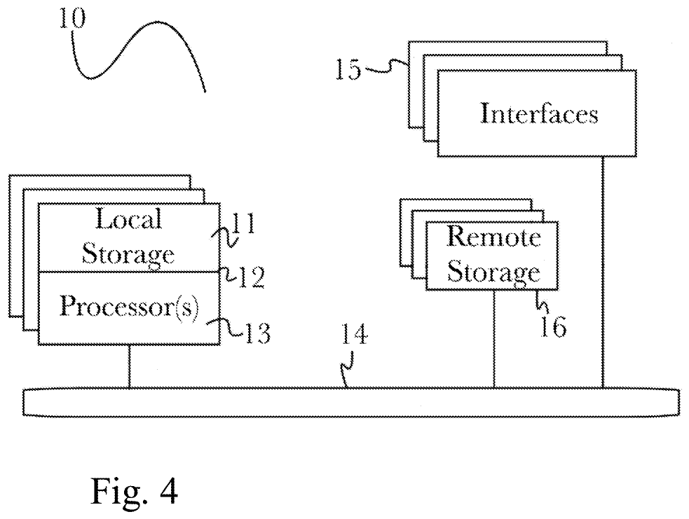

Referring now to FIG. 4, there is shown a block diagram depicting an exemplary computing device 10 suitable for implementing at least a portion of the features or functionalities disclosed herein. Computing device 10 may be, for example, any one of the computing machines listed in the previous paragraph, or indeed any other electronic device capable of executing software- or hardware-based instructions according to one or more programs stored in memory. Computing device 10 may be configured to communicate with a plurality of other computing devices, such as clients or servers, over communications networks such as a wide area network a metropolitan area network, a local area network, a wireless network, the Internet, or any other network, using known protocols for such communication, whether wireless or wired.

In one aspect, computing device 10 includes one or more central processing units (CPU) 12, one or more interfaces 15, and one or more busses 14 (such as a peripheral component interconnect (PCI) bus). When acting under the control of appropriate software or firmware, CPU 12 may be responsible for implementing specific functions associated with the functions of a specifically configured computing device or machine. For example, in at least one aspect, a computing device 10 may be configured or designed to function as a server system utilizing CPU 12, local memory 11 and/or remote memory 16, and interface(s) 15. In at least one aspect, CPU 12 may be caused to perform one or more of the different types of functions and/or operations under the control of software modules or components, which for example, may include an operating system and any appropriate applications software, drivers, and the like.

CPU 12 may include one or more processors 13 such as, for example, a processor from one of the Intel, ARM, Qualcomm, and AMD families of microprocessors. In some embodiments, processors 13 may include specially designed hardware such as application-specific integrated circuits (ASICs), electrically erasable programmable read-only memories (EEPROMs), field-programmable gate arrays (FPGAs), and so forth, for controlling operations of computing device 10. In a particular aspect, a local memory 11 (such as non-volatile random-access memory (RAM) and/or read-only memory (ROM), including for example one or more levels of cached memory) may also form part of CPU 12. However, there are many different ways in which memory may be coupled to system 10. Memory 11 may be used for a variety of purposes such as, for example, caching and/or storing data, programming instructions, and the like. It should be further appreciated that CPU 12 may be one of a variety of system-on-a-chip (SOC) type hardware that may include additional hardware such as memory or graphics processing chips, such as a QUALCOMM SNAPDRAGON™ or SAMSUNG EXYNOS™ CPU as are becoming increasingly common in the art, such as for use in mobile devices or integrated devices.

As used herein, the term “processor” is not limited merely to those integrated circuits referred to in the art as a processor, a mobile processor, or a microprocessor, but broadly refers to a microcontroller, a microcomputer, a programmable logic controller, an application-specific integrated circuit, and any other programmable circuit.

In one aspect, interfaces 15 are provided as network interface cards (NICs). Generally, NICs control the sending and receiving of data packets over a computer network; other types of interfaces 15 may for example support other peripherals used with computing device 10. Among the interfaces that may be provided are Ethernet interfaces, frame relay interfaces, cable interfaces, DSL interfaces, token ring interfaces, graphics interfaces, and the like. In addition, various types of interfaces may be provided such as, for example, universal serial bus (USB), Serial, Ethernet, FIREWIRE™, THUNDERBOLT™, PCI, parallel, radio frequency (RF), BLUETOOTH™, near-field communications (e.g., using near-field magnetics), 802.11 (WiFi), frame relay, TCP/IP, ISDN, fast Ethernet interfaces, Gigabit Ethernet interfaces, Serial ATA (SATA) or external SATA (ESATA) interfaces, high-definition multimedia interface (HDMI), digital visual interface (DVI), analog or digital audio interfaces, asynchronous transfer mode (ATM) interfaces, high-speed serial interface (HSSI) interfaces, Point of Sale (POS) interfaces, fiber data distributed interfaces (FDDIs), and the like. Generally, such interfaces 15 may include physical ports appropriate for communication with appropriate media. In some cases, they may also include an independent processor (such as a dedicated audio or video processor, as is common in the art for high-fidelity A/V hardware interfaces) and, in some instances, volatile and/or non-volatile memory (e.g., RAM).

Although the system shown in FIG. 4 illustrates one specific architecture for a computing device 10 for implementing one or more of the embodiments described herein, it is by no means the only device architecture on which at least a portion of the features and techniques described herein may be implemented. For example, architectures having one or any number of processors 13 may be used, and such processors 13 may be present in a single device or distributed among any number of devices. In one aspect, single processor 13 handles communications as well as routing computations, while in other embodiments a separate dedicated communications processor may be provided. In various embodiments, different types of features or functionalities may be implemented in a system according to the aspect that includes a client device (such as a tablet device or smartphone running client software) and server systems (such as a server system described in more detail below).

Regardless of network device configuration, the system of an aspect may employ one or more memories or memory modules (such as, for example, remote memory block 16 and local memory 11) configured to store data, program instructions for the general-purpose network operations, or other information relating to the functionality of the embodiments described herein (or any combinations of the above). Program instructions may control execution of or comprise an operating system and/or one or more applications, for example. Memory 16 or memories 11, 16 may also be configured to store data structures, configuration data, encryption data, historical system operations information, or any other specific or generic non-program information described herein.

Because such information and program instructions may be employed to implement one or more systems or methods described herein, at least some network device embodiments may include nontransitory machine-readable storage media, which, for example, may be configured or designed to store program instructions, state information, and the like for performing various operations described herein. Examples of such nontransitory machine-readable storage media include, but are not limited to, magnetic media such as hard disks, floppy disks, and magnetic tape; optical media such as CD-ROM disks; magneto-optical media such as optical disks, and hardware devices that are specially configured to store and perform program instructions, such as read-only memory devices (ROM), flash memory (as is common in mobile devices and integrated systems), solid state drives (SSD) and “hybrid SSD” storage drives that may combine physical components of solid state and hard disk drives in a single hardware device (as are becoming increasingly common in the art with regard to personal computers), memristor memory, random access memory (RAM), and the like. It should be appreciated that such storage means may be integral and non-removable (such as RAM hardware modules that may be soldered onto a motherboard or otherwise integrated into an electronic device), or they may be removable such as swappable flash memory modules (such as “thumb drives” or other removable media designed for rapidly exchanging physical storage devices), “hot-swappable” hard disk drives or solid state drives, removable optical storage discs, or other such removable media, and that such integral and removable storage media may be utilized interchangeably. Examples of program instructions include both object code, such as may be produced by a compiler, machine code, such as may be produced by an assembler or a linker, byte code, such as may be generated by for example a JAVA™ compiler and may be executed using a Java virtual machine or equivalent, or files containing higher level code that may be executed by the computer using an interpreter (for example, scripts written in Python, Perl, Ruby, Groovy, or any other scripting language).

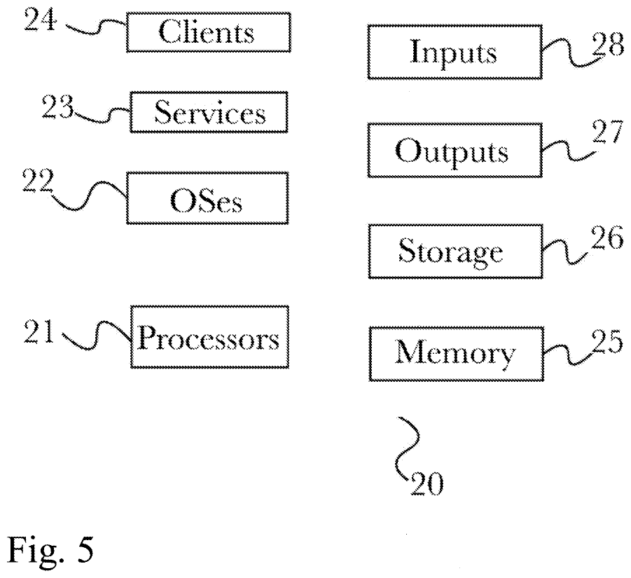

In some embodiments, systems may be implemented on a standalone computing system. Referring now to FIG. 5, there is shown a block diagram depicting a typical exemplary architecture of one or more embodiments or components thereof on a standalone computing system. Computing device 20 includes processors 21 that may run software that carry out one or more functions or applications of embodiments, such as for example a client application 24. Processors 21 may carry out computing instructions under control of an operating system 22 such as, for example, a version of MICROSOFT WINDOWS™ operating system, APPLE macOS™ or iOS™ operating systems, some variety of the Linux operating system, ANDROID™ operating system, or the like. In many cases, one or more shared services 23 may be operable in system 20, and may be useful for providing common services to client applications 24. Services 23 may for example be WINDOWS™ services, user-space common services in a Linux environment, or any other type of common service architecture used with operating system 21. Input devices 28 may be of any type suitable for receiving user input, including for example a keyboard, touchscreen, microphone (for example, for voice input), mouse, touchpad, trackball, or any combination thereof. Output devices 27 may be of any type suitable for providing output to one or more users, whether remote or local to system 20, and may include for example one or more screens for visual output, speakers, printers, or any combination thereof. Memory 25 may be random-access memory having any structure and architecture known in the art, for use by processors 21, for example to run software. Storage devices 26 may be any magnetic, optical, mechanical, memristor, or electrical storage device for storage of data in digital form (such as those described above, referring to FIG. 4). Examples of storage devices 26 include flash memory, magnetic hard drive, CD-ROM, and/or the like.

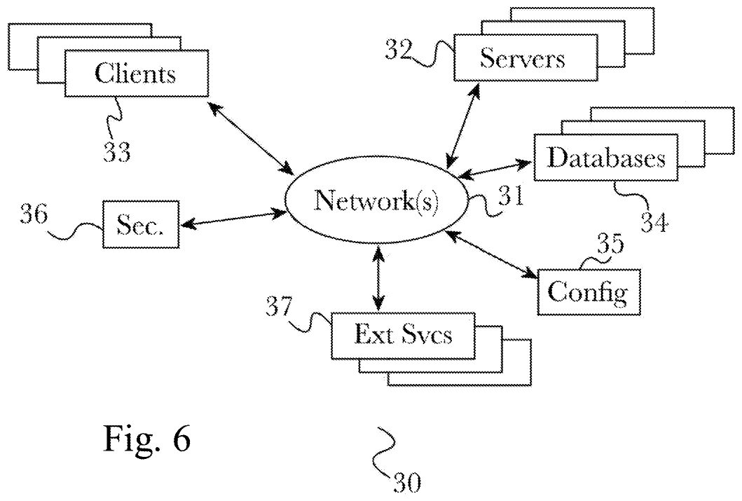

In some embodiments, systems may be implemented on a distributed computing network, such as one having any number of clients and/or servers. Referring now to FIG. 6, there is shown a block diagram depicting an exemplary architecture 30 for implementing at least a portion of a system according to one aspect on a distributed computing network. According to the aspect, any number of clients 33 may be provided. Each client 33 may run software for implementing client-side portions of a system; clients may comprise a system 20 such as that illustrated in FIG. 5. In addition, any number of servers 32 may be provided for handling requests received from one or more clients 33. Clients 33 and servers 32 may communicate with one another via one or more electronic networks 31, which may be in various embodiments any of the Internet, a wide area network, a mobile telephony network (such as CDMA or GSM cellular networks), a wireless network (such as WiFi, WiMAX, LTE, and so forth), or a local area network (or indeed any network topology known in the art; the aspect does not prefer any one network topology over any other). Networks 31 may be implemented using any known network protocols, including for example wired and/or wireless protocols.

In addition, in some embodiments, servers 32 may call external services 37 when needed to obtain additional information, or to refer to additional data concerning a particular call. Communications with external services 37 may take place, for example, via one or more networks 31. In various embodiments, external services 37 may comprise web-enabled services or functionality related to or installed on the hardware device itself. For example, in one aspect where client applications 24 are implemented on a smartphone or other electronic device, client applications 24 may obtain information stored in a server system 32 in the cloud or on an external service 37 deployed on one or more of a particular enterprise's or user's premises.

In some embodiments, clients 33 or servers 32 (or both) may make use of one or more specialized services or appliances that may be deployed locally or remotely across one or more networks 31. For example, one or more databases 34 may be used or referred to by one or more embodiments. It should be understood by one having ordinary skill in the art that databases 34 may be arranged in a wide variety of architectures and using a wide variety of data access and manipulation means. For example, in various embodiments one or more databases 34 may comprise a relational database system using a structured query language (SQL), while others may comprise an alternative data storage technology such as those referred to in the art as “NoSQL” (for example, HADOOP CASSANDRA™, GOOGLE BIGTABLE™, and so forth). In some embodiments, variant database architectures such as column-oriented databases, in-memory databases, clustered databases, distributed databases, or even flat file data repositories may be used according to the aspect. It will be appreciated by one having ordinary skill in the art that any combination of known or future database technologies may be used as appropriate, unless a specific database technology or a specific arrangement of components is specified for a particular aspect described herein. Moreover, it should be appreciated that the term “database” as used herein may refer to a physical database machine, a cluster of machines acting as a single database system, or a logical database within an overall database management system. Unless a specific meaning is specified for a given use of the term “database”, it should be construed to mean any of these senses of the word, all of which are understood as a plain meaning of the term “database” by those having ordinary skill in the art.

Similarly, some embodiments may make use of one or more security systems 36 and configuration systems 35. Security and configuration management are common information technology (IT) and web functions, and some amount of each are generally associated with any IT or web systems. It should be understood by one having ordinary skill in the art that any configuration or security subsystems known in the art now or in the future may be used in conjunction with embodiments without limitation, unless a specific security 36 or configuration system 35 or approach is specifically required by the description of any specific aspect.

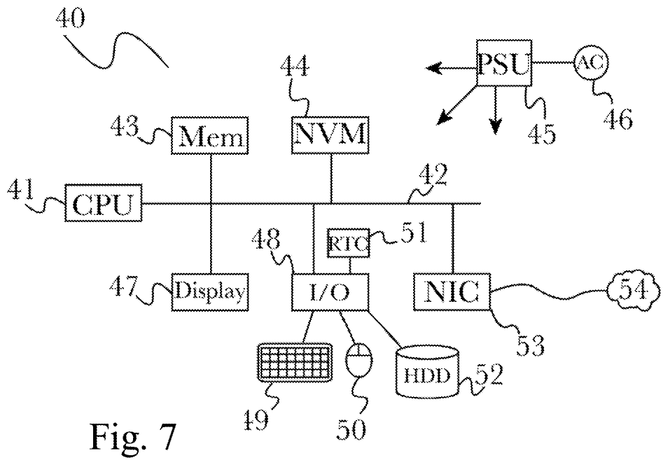

FIG. 7 shows an exemplary overview of a computer system 40 as may be used in any of the various locations throughout the system. It is exemplary of any computer that may execute code to process data. Various modifications and changes may be made to computer system 40 without departing from the broader scope of the system and method disclosed herein. Central processor unit (CPU) 41 is connected to bus 42, to which bus is also connected memory 43, nonvolatile memory 44, display 47, input/output (I/O) unit 48, and network interface card (NIC) 53. I/O unit 48 may, typically, be connected to keyboard 49, pointing device 50, hard disk 52, and real-time clock 51. NIC 53 connects to network 54, which may be the Internet or a local network, which local network may or may not have connections to the Internet. Also shown as part of system 40 is power supply unit 45 connected, in this example, to a main alternating current (AC) supply 46. Not shown are batteries that could be present, and many other devices and modifications that are well known but are not applicable to the specific novel functions of the current system and method disclosed herein. It should be appreciated that some or all components illustrated may be combined, such as in various integrated applications, for example Qualcomm or Samsung system-on-a-chip (SOC) devices, or whenever it may be appropriate to combine multiple capabilities or functions into a single hardware device (for instance, in mobile devices such as smartphones, video game consoles, in-vehicle computer systems such as navigation or multimedia systems in automobiles, or other integrated hardware devices).

In various embodiments, functionality for implementing systems or methods of various embodiments may be distributed among any number of client and/or server components. For example, various software modules may be implemented for performing various functions in connection with the system of any particular aspect, and such modules may be variously implemented to run on server and/or client components.

The skilled person will be aware of a range of possible modifications of the various embodiments described above. Accordingly, the present invention is defined by the claims and their equivalents.

### Additional Considerations

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the invention. This description should be read to include one or at least one and the singular also includes the plural unless it is obvious that it is meant otherwise.

Upon reading this disclosure, those of skill in the art will appreciate still additional alternative structural and functional designs for a system and a process for creating an interactive message through the disclosed principles herein. Thus, while particular embodiments and applications have been illustrated and described, it is to be understood that the disclosed embodiments are not limited to the precise construction and components disclosed herein. Various apparent modifications, changes and variations may be made in the arrangement, operation and details of the method and apparatus disclosed herein without departing from the spirit and scope defined in the appended claims.

What is claimed is:

1. A computer implemented method for enabling real-time access to standardized medication information, the computer implemented method comprising:
providing remote access to users over a network so any one of the users can provide medication information and medical insurance information in real time through a graphical user interface, wherein the medication information and/or medical insurance information is provided in at least one of standardized and non-standardized format based on hardware and/or software platform used by at least one of the users;
obtaining medication information from the at least one of the users, wherein the medication information comprises at least one of a medication name and dose information, wherein the medication information is comprised of a medication class;
obtaining medical insurance information from the user;
retrieving formulary information associated with a plurality of medical insurance plans, wherein the formulary information comprises at least one of a list of covered medications, covered dose information associated with covered medications, and covered diagnoses or diagnosis codes, wherein the retrieved formulary information associated with the plurality of medical insurance plans is in a non-standardized format;
converting, by a processor, the formulary information associated with a plurality of medical insurance plans into a standardized format;
storing, in a formulary database, the standardized format of the formulary information;
automatically determining, by a processor, whether the obtained medication information is covered by the medical insurance plan by performing a lookup operation in the at least one of the formulary database and the retrieved formulary information;
identifying at least one alternative medication to the obtained medication information, wherein the identified at least one alternative medication is listed in the formulary database in association with the obtained medical insurance information, and wherein identifying at least one alternative medication comprises identifying at least one medication having a medication class that is the same as a medication class of the obtained medication information;
automatically converting, by a processor, dose information associated with the at least one identified alternative medication, wherein the dose information for the at least one alternative medication is equivalent to the obtained medication dose information; and
providing the at least one identified alternative medication and converted dose information via remote access to users over a network, wherein each user is enabled to access up-to-date at least one identified alternative medication and converted dose information in the standardized format.

2. The computer implemented method according to claim 1, wherein dose information comprises at least one of dose, dosage, dosage form, and dosage regimen associated with the prescribed medication.

3. The computer implemented method according to claim 1, wherein the medical insurance information comprises at least one of medical insurance company name, medical insurance plan name, plan benefit level, and medical insurance identifier associated with a patient.

4. The computer implemented method according to claim 1, wherein at least one of the medication information and medical insurance information is obtained from an electronic medical record or electronic health record.

5. The computer implemented method according to claim 1, wherein the formulary information further comprises covered medication classes.

6. The computer implemented method according to claim 1, wherein the formulary information is retrieved from at least one of a database storing formulary information or a website associated with a medical insurance company associated with the medical insurance information.

7. The computer implemented method according to claim 6, wherein the database is updated at threshold time intervals or when new formulary information is provided by a medical insurance company.

8. The computer implemented method according to claim 1, wherein the medication class comprises medications having a same or similar mechanism of action or medications for treating a same diagnosis.

9. The computer implemented method according to claim 1, further comprising receiving, from a prescribing practitioner, an indication confirming or declining the alternative medication and dose information.

10. The computer implemented method according to claim 1, further comprising communicating the obtained medication information and dose information or the alternative medication and converted dose information to an indicated medication dispenser or medication fulfillment entity.

11. The computer implemented method according to claim 1, further comprising determining that the obtained medication information is not covered by the medical insurance plan when the obtained medication information is not listed in the formulary information, the determining comprising comparing the obtained medication information with the formulary information in the standardized format to identify the absence of the medication information in the formulary information.

12. The computer implemented method according to claim 11, further comprising providing an indication to the user or associated personnel, in real time, when the obtained medication information is determined to be not covered.

13. The computer implemented method according to claim 1, further comprising determining that the obtained medication information is covered by the medical insurance plan when the obtained medication information is listed in the formulary information, the determining comprising comparing the obtained medication information with the formulary information in the standardized format to identify the presence of the medication information in the formulary information.

14. The computer implemented method according to claim 13, further comprising providing an indication to the user or associated personnel, in real time, when the obtained medication information is determined to be covered.

15. The computer implemented method according to claim 1, wherein identifying an alternative medication is only performed in response to determining, by a processor, that the obtained medication information is not covered in association with the obtained medical insurance information.

16. The computer implemented method according to claim 1, wherein the medication information is provided in a non-standardized format; and further comprising converting, by a processor, the medication information into a standardized format;
and wherein the automatically determining whether the obtained medication information is covered by the medical insurance plan comprises comparing the medication information in the standardized format with the standardized format of the formulary information.

17. The computer implemented method according to claim 1, wherein at least one of the medication information and medical insurance information is provided by a healthcare practitioner and is associated with a specific patient.

18. The computer implemented method according to claim 1, wherein the users comprise at least one of a patient, a healthcare practitioner, a medication dispenser, and medication fulfillment entity.

19. A system for enabling real-time access to standardized medication information, the system comprising:
control circuitry configured to perform a method comprising:
providing remote access to users over a network so any one of the users can provide medication information and medical insurance information in real time through a graphical user interface, wherein the medication information and/or medical insurance information is provided in at least one of standardized and non-standardized format based on hardware and/or software platform used by at least one of the users;
obtaining medication information from the at least one of the users, wherein the medication information comprises at least one of a medication name and dose information, wherein the medication information is comprised of a medication class;
obtaining medical insurance information from the user;
retrieving formulary information associated with a plurality of medical insurance plans, wherein the formulary information comprises at least one of a list of covered medications, covered dose information associated with covered medications, and covered diagnoses or diagnosis codes, wherein the retrieved formulary information associated with the plurality of medical insurance plans is in a non-standardized format;
converting, by a processor, the formulary information associated with a plurality of medical insurance plans into a standardized format;
storing, in a formulary database, the standardized format of the formulary information;
automatically determining, by a processor, whether the obtained medication information is covered by the medical insurance plan by performing a lookup operation in the at least one of the formulary database and the retrieved formulary information;
identifying at least one alternative medication to the obtained medication information, wherein the identified at least one alternative medication is listed in the formulary database in association with the obtained medical insurance information, and wherein identifying at least one alternative medication comprises identifying at least one medication having a medication class that is the same as a medication class of the obtained medication information;
automatically converting, by a processor, dose information associated with the at least one identified alternative medication, wherein the dose information for the at least one alternative medication is equivalent to the obtained medication dose information; and
providing the at least one identified alternative medication and converted dose information via remote access to users over a network, wherein each user is enabled to access up-to-date at least one identified alternative medication and converted dose information in the standardized format.

20. A non-transitory computer readable medium comprising instructions that, when executed by a processor, cause the processor to perform a method for enabling real-time access to standardized medication information, the method comprising:
providing remote access to users over a network so any one of the users can provide medication information and medical insurance information in real time through a graphical user interface, wherein the medication information and/or medical insurance information is provided in at least one of standardized and non-standardized format based on hardware and/or software platform used by at least one of the users;
obtaining medication information from the at least one of the users, wherein the medication information comprises at least one of a medication name and dose information, wherein the medication information is comprised of a medication class;
obtaining medical insurance information from the user;
retrieving formulary information associated with a plurality of medical insurance plans, wherein the formulary information comprises at least one of a list of covered medications, covered dose information associated with covered medications, and covered diagnoses or diagnosis codes, wherein the retrieved formulary information associated with the plurality of medical insurance plans is in a non-standardized format;
converting, by a processor, the formulary information associated with a plurality of medical insurance plans into a standardized format;
storing, in a formulary database, the standardized format of the formulary information;
automatically determining, by a processor, whether the obtained medication information is covered by the medical insurance plan by performing a lookup operation in the at least one of the formulary database and the retrieved formulary information;
identifying at least one alternative medication to the obtained medication information, wherein the identified at least one alternative medication is listed in the formulary database in association with the obtained medical insurance information, and wherein identifying at least one alternative medication comprises identifying at least one medication having a medication class that is the same as a medication class of the obtained medication information;
automatically converting, by a processor, dose information associated with the at least one identified alternative medication, wherein the dose information for the at least one alternative medication is equivalent to the obtained medication dose information; and
providing the at least one identified alternative medication and converted dose information via remote access to users over a network, wherein each user is enabled to access up-to-date at least one identified alternative medication and converted dose information in the standardized format.
